(19) United States
(12) Patent Application Publication
(10) Pub. No.: US 2006/0064045 A1
(43) Pub. Date: Mar. 23, 2006

(54) WEARABLE BODY SUPPORT SYSTEM

(76) Inventor: Mahyar Khavari, Valley Cottage, NY (US)

Correspondence Address: DICKINSON WRIGHT PLLC, 1901 L. STREET NW, SUITE 800, WASHINGTON, DC, 20036, US

(21) Appl. No.: 10/711,464

(22) Filed: Sep. 20, 2004

(51) Int. Cl. A61F 5/00

(52) U.S. Cl. 602/19; 602/12

(57) ABSTRACT

A wearable body support system includes a first elongated cushion assembly and a second elongated cushion assembly and an upper strap attached to a top portion of the first and second elongated cushion assemblies configured to attach to a user's waist. The first and second elongated cushion assemblies are separate components and are configured to be worn by a user and provide cushioning and support to the user's posterior lower torso and legs. The wearable body support may also include an upper torso support configured to be worn by a user and provide support to the user's back. The upper torso support may also be configured with a buoyant material to be further used as a life vest. A pair of lower leg supports may also be included to provide support to the user's posterior lower legs.

### FIELD OF THE INVENTION

The present invention generally relates to a wearable body support system and particularly to a wearable body support system configured for attachment to a person that provides cushioning and support to the user during events such as hunting, fishing, boating, rafting, spectator events and the like.

### DISCUSSION OF RELATED ART

During activities such as boating, sailing, hunting and fishing, individuals may be forced to sit on hard or uncomfortable surfaces for long periods of time. For example, sailing may require a person to remain in a certain location on the sailboat and may have to sit on uncomfortable objects or surfaces to keep the boat from heeling in high winds. Although there are many different types of portable cushions that may be used during this and other types of activities, they do not provide an optimal solution to the user. The user may have to use his hands to hold onto other necessary objects and may not have the available hand to carry a separate cushion. Further, as the person moves from a sitting to standing position, or vice versa, a cushion may slip from its placed position.

To help solve the above-mentioned problems portable seat cushions and seating units have been available for spectators to take to events such as sporting events, or for sportsmen to use during activities such as hunting and fishing. These types of seating units have typically been a simple square shaped seat or cushion that the user carries to the event or activity and positions on the object for which the individual is to sit, providing a more comfortable experience during the particular event or activity. Some such seating units may even additionally provide a seat back to provide further comfort to the user. However, adding a back support component to the seating unit may increase the weight of the unit and make it more difficult to carry to the event or activity. Other portable seat cushions provide straps to assist the user in carrying the seating unit.

In recent years more complicated portable seating arrangements have been developed that include the ability to attach the seating unit to the user's body. One such configuration is a contoured body cushion described in U.S. Pat. No. 6,357,829 to Hanke. The cushion discussed in Hanke is fastenable to the user's body through straps positioned at the waist and legs of the user. The Hanke cushion is a one-piece construction with extending leg portions integral with a lower torso portion. Unfortunately, Hanke does not provide any support to the upper torso of the user. Further, the integral construction of the Hanke cushion may limit the flexibility and mobility of the cushion when worn by the user.

Another type of portable seating unit is the wearable seat described in U.S. Pat. No. 6,347,406 issued to Jones et al. The Jones et al. system is attached to a vest configured to be worn by the user. The seating portion is folded to an up position during transit and then can be manually unfolded and placed on the ground while the vest remains on the user. The Jones et al. seat system does not provide cushioning to the user nor does it provide a seat or cushion that is flexible and moveable as the user changes from a standing position to a sitting position. In fact, the user would have to fold the seat back to the up or stowed positioned to walk or move to another location.

Thus, there is a desire and need in the art to provide a usable and practical wearable body support system configured to attach to a person and that is constructed of flexible and lightweight materials to allow maximum mobility of the user. There is a further need for a wearable body support that provides cushioning or support to both the upper and lower torso as well as the legs of the user.

### SUMMARY OF THE INVENTION

Accordingly, the present invention provides a wearable body support system configured for attachment to a user's body that provides cushioning and support to the user for use during events such as hunting, fishing, boating, rafting, spectator sporting events and the like. The wearable body support system is constructed with flexible and lightweight components to allow maximum mobility to the user and allowing the user to participate in a variety of activities while wearing the support system.

In one embodiment of the present invention a wearable body support system includes a first elongated cushion assembly and a second elongated cushion assembly. An upper strap is attached to a top portion of the first and second elongated cushion assemblies and is configured to attach to a user's waist. The first and second elongated cushion assemblies are separate components and are configured to be worn by a user and provide cushioning and support to the user's posterior lower torso and legs.

In another embodiment of the present invention a wearable body support system includes a first elongated cushion assembly and a second elongated cushion assembly and an upper strap attached to a top portion of the first and second elongated cushion assemblies. The upper strap is configured to attach to a user's waist. A connecting strap extends between and connects a top portion of the first elongated cushion assembly and a top portion of the second elongated cushion assembly. The first elongated cushion assembly and said second elongated cushion assembly are configured to attach to a user's body and extend from the user's lower torso to the user's legs and provide support to the user's posterior lower torso and legs.

In yet another embodiment of the present invention a wearable body support system includes an upper torso support having an upper cushion assembly and a pair of arm straps attached to the cushion assembly. A lower torso support is included and has a first elongated cushion assembly and a second elongated cushion assembly and an upper strap attached to a top portion of the first and second elongated cushion assemblies. The upper torso support is configured to attach to a user's upper body and provide support to the user's back. The lower torso support is configured to attach to a user's lower torso and legs and provide support to the user's posterior lower torso and legs.

Other features of the present invention will become more apparent to persons having ordinary skill in the art to which the present invention pertains from the following description and claims taken in conjunction with the accompanying figures.

### BRIEF DESCRIPTION OF THE FIGURES

The foregoing features, as well as other features, will become apparent with reference to the description and figures below, in which like numerals represent like elements, and in which:

FIG. 1 is front view of a wearable body support of the present invention shown in an unclasped position;

FIG. 2 is front view of a wearable body support of the present invention shown in a clasped position;

FIG. 3 is a front perspective view of a portion of a typical cushion assembly of the present invention;

FIG. 4 is a side view of a wearable body support of the present invention shown as attached to a user;

FIG. 5 is a side view of a wearable body support of the present invention shown with an optional knee support; and

FIG. 6 is an embodiment of a wearable body support of the present invention.

### DETAILED DESCRIPTION

The present invention provides a wearable body support system configured to attach to the user and provide a convenient and comfortable seating unit that the user can wear and use at virtually any location. The wearable body support system is constructed with flexible materials and components to allow for maximum mobility to the user. Because of the simple and light weight construction of the wearable body support system, it is ideal for use at events and activities such as hunting, fishing, rafting boating, hiking, attending spectator sports events and the like. The wearable body support system also provides a variety of options to add to the appearance and versatility of the wearable body support system such as adding logos and advertising and can also be constructed in a variety of sizes and shapes. Additional cushioning components may also be added, such as knee supports, to allow further comfort to a user that may need to kneel on the ground.

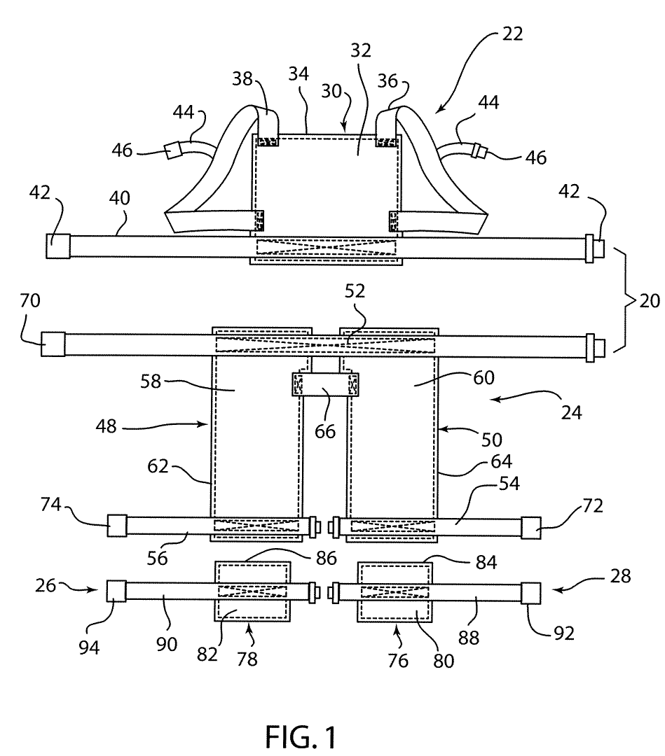

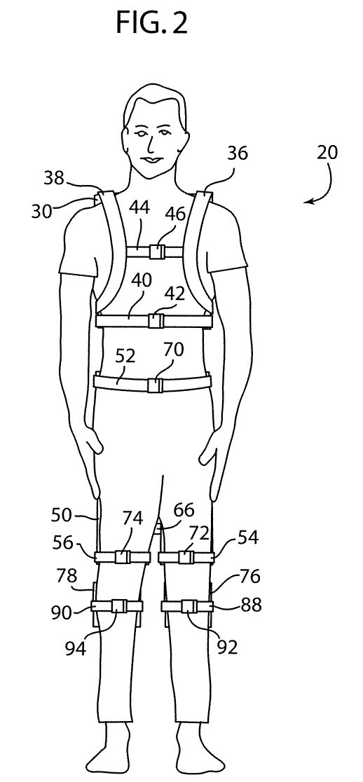

Referring now to FIGS. 1 and 2, a wearable body support system 20 may include an upper torso support 22, a lower torso support 24, and two lower leg supports 26 and 28. Upper torso support 22 is configured to be worn by a user on his upper torso to provide cushioning and support to the user's back. Such support may be desired when a user participates in an activity or event where he may need to lean back against an object. For example, a hunter may need to lean back against a tree trunk. Upper torso support 22 includes an upper cushion assembly 30 having a padding element 32 and an optional cover 34. Upper torso support 22 may also include first and second arm straps 36 and 38 attached to cushion assembly 30 to use to secure upper torso support 22 to the user's upper body. Although not required, a bottom strap 40 may also be attached to cushion assembly 30 to further secure upper torso support 22 to the user.

Padding element 32 may be constructed with a variety of lightweight materials such as various closed cell foam materials and may alternatively include a buoyant material as used in life jackets to allow upper torso support 22 to be used as a life vest. Cover 34 may also be constructed with a variety of materials such as cloth fabric, vinyl, nylon or other available materials and preferably is constructed with a wear and scuff resistant material to improve durability of wearable body support system 20. Cover 34 is configured to fit over and conceal padding element 32, and may be sewn closed over padding element 32 or alternatively may be closed with a zipper closure, VELCRO, snaps or other known fastening methods.

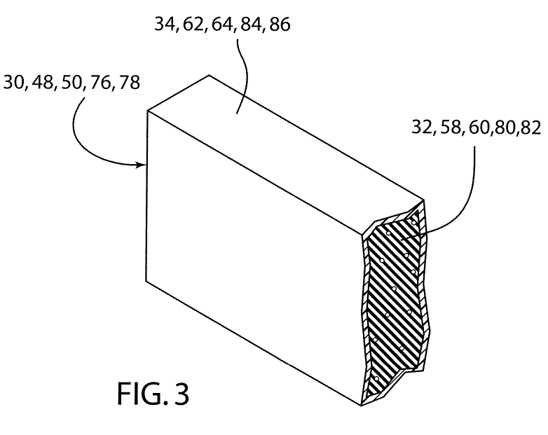

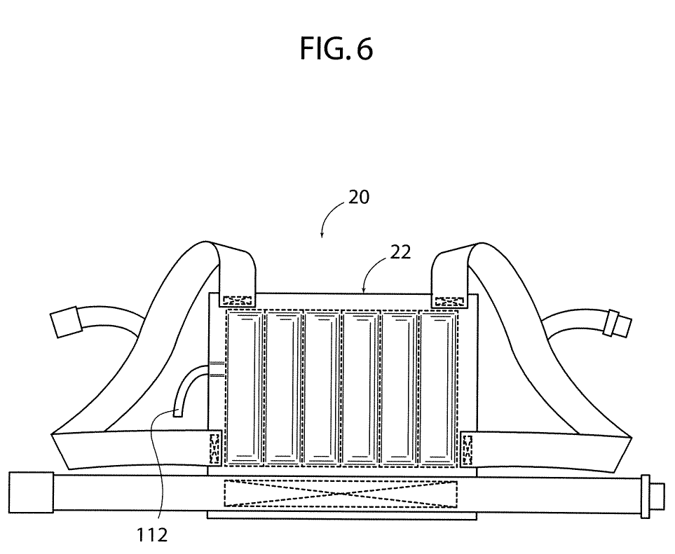

The illustrated construction of cushion assembly 30 is representative of all possible cushion assemblies included with wearable body support 20 and is most easily viewed in FIG. 3. The cushion assemblies are shown with a foam element enclosed within a cover. However, other optional features may be included. For example, an inflatable tube element 112 may be included to allow the user to inflate the cushion assembly to a desired cushioning level as shown in FIG. 6.

It is also contemplated that all the straps utilized to secure the various supports to the user would be constructed in an identical fashion. Further, the straps and corresponding fasteners preferably allow for size adjustment utilizing typical slide features to allow the strap to be shortened or lengthened as needed.

Arm straps 36 and 38, chest strap 44 and optional bottom strap 40 may be constructed with fabric, such as a durable cotton, or nylon, plastic, or other suitable material used for typical straps or belts. Arm straps 36 and 38 may be sewn into cushion assembly 30, or attached by other known methods for similar components. Arm straps 36 and 38 may be further connected with a chest strap 44 having a fastener 46. Chest strap 44 may be sewn to arm straps 36 and 38 on opposite ends as shown in FIGS. 1 and 2, and is used to securely hold upper torso support 22 on the user's upper body. Bottom strap 40 may be constructed and attached to cushion assembly 30 the same as arm straps 36 and 38. Bottom strap 40 also includes a fastener 42 similar to fastener 46. Fasteners 42 and 46 may include any of a variety of fasteners such as a typical snap fit clasp, clips, VELCRO, ties, or belt type fastener and may be a variety of suitable sizes to meet the needs of the user.

In use, the user places his arms through arm straps 36 and 38 and fastens fastener 46. Cushion assembly 30 will be positioned against the user's back to provide comfort and support to the user when the user needs to lean back against a hard surface. In an embodiment having a bottom strap 40, the user would secure bottom strap 40 around his upper torso and fasten fastener 42.

Lower torso support 24 is configured to be worn by the user and provide cushioning and support to the user's posterior lower body, including his lower torso and legs. Lower torso support 24 may include a first and second elongated cushion assembly 48 and 50, an upper strap 52, and first and second leg straps 54 and 56.

First and second elongated cushion assemblies 48 and 50 may each include a padding element 58, 60 and a cover 62, 64. First and second elongated cushion assemblies 48 and 50 may be constructed with the same materials as upper cushion assembly 30 described above. Upper strap 52 and leg straps 54 and 56 may be sewn to elongated cushion assemblies 48 and 50 or attached by other known means as shown in FIGS. 1 and 2. Lower torso support 24 may also include an upper connecting strap 66 to connect elongated cushion assemblies 48 and 50 at an upper location such that they are able to move independently yet remain securely in position on the user's body. Leg straps 54 and 56 are configured to secure first and second elongated cushion assemblies 48 and 50 to the user's legs. Fasteners 70, 72 and 74 are used to secure straps 52, 54 and 56 respectively, and may be the type of fastener as fasteners 42 and 46 described above.

In use, the user would position first and second elongated cushion assemblies 48 and 50 on the posterior lower torso and upper legs of the user and secure upper strap 52 around the user's waist by fastening fastener 70. The user would also secure leg straps 54 and 56 around the user's legs by fastening fasteners 72 and 74.

Wearable body support system 20 may also include first and second lower leg supports 26 and 28 that are similarly constructed using techniques known in the art as upper and lower torso supports 22 and 24. Lower leg supports 26 and 28 may include a cushion assembly 76 and 78 having a padding element 80 and 82 and a cover 84 and 86. Lower leg supports 26 and 28 may each also include a lower leg strap 88 and 90 and a fastener 92 and 94. Lower leg sections 26 and 28 are configured to attach to the user's lower legs to provide further comfort and support to the user.

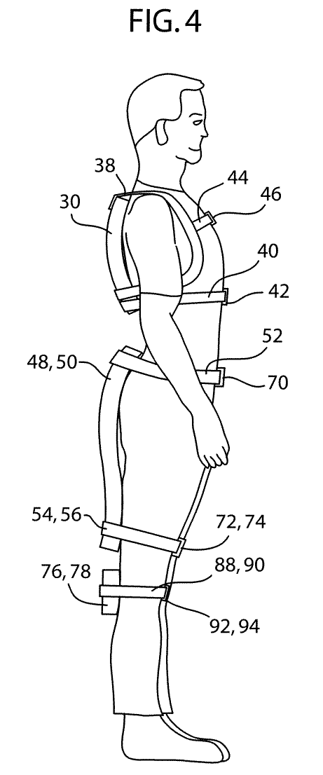

As shown in FIGS. 2 and 4, the various supports included with wearable body support system 20 may be attached to the user's body. The flexible and lightweight construction allows the user to participate in a variety of activities while wearing support system 20. One or all of the described portions of the support system 20 may be worn at one time, depending on the needs of the user.

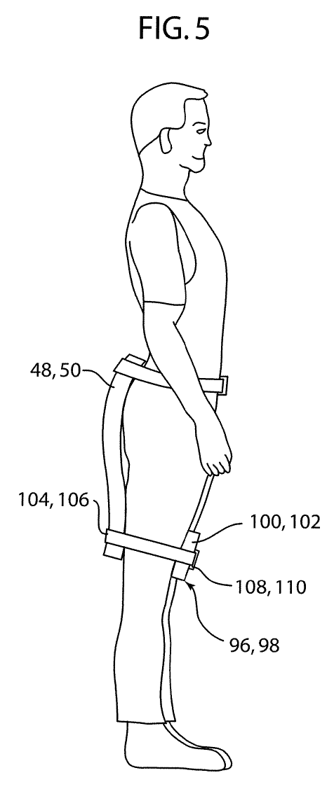

An optional knee support 96, 98 may be included to provide further comfort when the user is required to kneel as shown in FIG. 5. Knee supports 96, 98 may include a cushion assembly 100, 102 attached to a knee strap 104, 106. Knee straps 104, 106 may also be attached to first and second elongated cushion assemblies 48 and 50 with typical attachment methods as described above, and include a fastener 108, 110 to secure knee support sections 96 and 98 to the user's knees. Knee supports 96 and 98 may be constructed with the same materials as described above for upper torso support 22, lower torso support 24 and leg supports 26 and 28.

There are other optional features that may be added to wearable body support system 20 such as advertising logos or emblems, or other graphics. In marine applications options might include attachments for whistles, mirrors, flares, inflatable vest, reflecting tape, strobe flashlights, side pockets, and the like. A variety of different colors may be used to meet the particular style or needs of the user, such as orange or camouflage for use in hunting. The various supports included in wearable body support system 20 may also be a variety of different sizes to accommodate both children and adults.

While the invention has been described in conjunction with specific embodiments, it is evident that many alternatives, modifications and variations will be apparent to those skilled in the art in light of the foregoing description. Accordingly, the present invention attempts to embrace all such alternatives, modifications and variations that fall within the spirit and scope of the appended claims.

1. A wearable body support system, comprising:
a first elongated cushion assembly and a second elongated cushion assembly;
an upper strap attached to a top portion of said first and second elongated cushion assemblies and configured to attach to a user's waist;
wherein said first elongated cushion assembly and said second elongated cushion assembly are separate components and are configured to be, worn by a user and provide cushioning and support to the user's posterior lower torso and legs;
an upper torso support having an upper cushion assembly, a pair of arm straps attached to said upper cushion assembly and a bottom strap attached to a bottom portion of said upper cushion assembly; and
wherein said upper torso support is configured to attach to a user's upper torso and provide cushioning and support to the user's back;
said upper cushion assembly includes a padding element; and
wherein said padding element in said upper cushion assembly includes a buoyant material.

2. (canceled)

3. The wearable body support system of claim 1, further comprising:
a first lower leg support and a second lower leg support;
said first and second leg support including, a lower leg cushion assembly and a lower leg strap attached to said lower leg cushion assembly and a lower leg strap attached to said lower leg cushion assembly; and
wherein said first and second lower leg supports are configured to attach to the user's lower legs.

4. The wearable body support system of claim 1, further comprising:
a connecting strap extending between and connecting a top portion of said first elongated cushion assembly and a top portion of said second elongated cushion assembly.

5. The wearable body support system of claim 1, further comprising:
a first knee support attached to said first elongated cushion assembly and a second knee support attached to said second elongated cushion assembly.

6. The wearable body support system of claim 1, wherein said first and second elongated cushion assemblies include a padding element and a cover.

7. (canceled)

8. (canceled)

9. The wearable body support system of claim 1, wherein said upper cushion assembly includes an inflatable tube element.

10. The wearable body support system of claim 6, wherein said cover includes a graphic.

11. A wearable body support system, comprising:
a first elongated cushion assembly and a second elongated cushion assembly;
an upper strap attached to a top portion of said first and second elongated cushion assemblies configured to attach to a user's waist;
a connecting strap extending between and connecting a top portion of said first elongated cushion assembly and a top portion of said second elongated cushion assembly;
wherein said first elongated cushion assembly and said second elongated cushion assembly are configured to attach to a user's body extending from the user's lower torso to the user's legs and provide support to the user's posterior lower torso and legs;
an upper torso support having an upper cushion assembly, a pair of arm straps attached to said upper cushion assembly and a bottom strap attached to a bottom portion of said upper cushion assembly; and
wherein said upper torso support is configured to attach to a user's upper torso and provide cushioning and support to the user's back.

12. (canceled)

13. The wearable body support system of claim 11, further comprising:
a first lower leg support and a second lower leg support; said first and second leg support including a lower leg cushion assembly and a lower leg strap attached to said lower leg cushion assembly; and
wherein said first and second lower leg supports are configured to attach to the user's lower legs and provide support to the user's posterior lower leg.

14. The wearable body support system of claim 11, further comprising:
a first knee support attached to said first elongated cushion assembly and a second knee support attached to said second elongate cushion assembly.

15. The wearable body support system of claim 11, wherein said first and second elongated cushion assemblies include a padding element and a cover.

16. The wearable body support system of claim 11, wherein said upper cushion assembly includes a padding element and a cover.

17. The wearable body support system of claim 16, wherein said padding element includes a buoyant material.

18. The wearable body support system of claim 11, wherein said upper cushion assembly includes an inflatable tube element.

19. The wearable body support system of claim 15, wherein said cover includes a graphic.

20. A wearable body support system, comprising:
an upper torso support having an upper cushion assembly and a pair of arm straps attached to said cushion assembly;
a lower torso support having a first elongated cushion assembly and a second elongated cushion assembly, an upper strap attached to a top portion of said first and second elongated cushion assemblies; and
wherein said upper torso support is configured to attach to a user's upper body to support a user's back and said lower torso support is configured to attach to a user's lower torso and legs and provide support to the user's posterior lower torso and legs.

21. The wearable body support system of claim 20, further comprising:
a first lower leg support and a second lower leg support; said first and second leg support including a lower leg cushion assembly and a lower leg strap attached to said lower leg cushion assembly; and
wherein said first and second lower leg supports are configured to attach to the user's lower legs.

22. The wearable body support system of claim 20, wherein said upper cushion assembly includes a padding element and a cover.

23. The wearable body support system of claim 22, wherein said padding element includes a buoyant material.

24. The wearable body support system of claim 20, further comprising a first knee support attached to said first elongated cushion assembly and a second knee support attached to said second elongated cushion assembly.
